(19) United States
(12) Patent Application Publication
(10) Pub. No.: US 2011/0009651 A1
(43) Pub. Date: Jan. 13, 2011

(54) REGENERATION METHOD OF TITANOSILICATE CATALYST

(75) Inventors: Tomonori Kawabata, Osaka (JP); Hiroaki Abekawa, Osaka (JP); Makoto Yako, Wakayama (JP)

Correspondence Address: FOLEY AND LARDNER LLP;SUITE 500, 3000 K STREET NW, WASHINGTON, DC, 20007, US

(73) Assignee: Sumitomo Chemical Company, Limited

(21) Appl. No.: 12/921,130

(22) PCT Filed: Mar. 3, 2009
(86) PCT No.: PCT/JP2009/054589

§ 371 Date: Sep. 3, 2010

(30) Foreign Application Priority Data
Mar. 5, 2008 (JP) 2008-954511

(51) Int. Cl. C07D 301/04; B01J 38/50; B01J 38/02; B01J 38/00

(52) U.S. Cl. 549/523; 502/29; 502/56; 502/20

(57) ABSTRACT

The present invention provides a regeneration method of a titanosilicate catalyst, specifically provides a regeneration method of a titanosilicate catalyst which comprises contacting the titanosilicate catalyst deteriorated in catalytic ability with a nitrile compound or a mixture of water and a nitrile compound at a temperature from 25° C. to 200° C.

### TECHNICAL FIELD

The present invention relates to a regeneration method of a titanosilicate catalyst which has been deteriorated in the catalytic ability.

### BACKGROUND ART

It has been conventionally known that titanosilicate is usable as a catalyst in the synthesis reaction of propylene oxide from propylene, oxygen and hydrogen. As a method of regenerating a used titanosilicate catalyst, a method has been suggested in which TS-1 catalyst supported on palladium (Pd/TS-1) used in the synthesis reaction of propylene oxide from propylene, oxygen and hydrogen is contacted with water or an alcohol or a water/alcohol mixed solvent and thereby regenerated, and a method of activating the catalyst using a water/alcohol mixed solvent at 60 to 150° C. has been exemplified (see WO2006001876A1).

It is known that when the regenerating method using an alcohol alone or a water/alcohol mixed solvent is applied to a reaction system using a solvent other than methanol in the reaction, as in a case that the method is applied to the synthesis reaction of propylene oxide using a Ti-MWW catalyst, for example, in an acetonitrile solvent, the activity is deteriorated by the addition of methanol to the reaction system (for example, see Heisei 13 Nen-do Jisedai Kagaku Process Gijutsu Kaihatsu and Non-halogen Kagaku Kagaku Process Gijutsu.Kaihatsu Seika Houkokusho (Achievement Report for the Year 2001 of Next Generation Chemistry Process Technology Development and Non-halogen Chemistry Process Technology Development), 168-210 (2002)).

When methanol is used as an alcohol, methanol readily reacts with propylene oxide and generates methoxy propanol, and thus use of an alcohol may cause by-production of an alkoxy propanol and is not necessarily industrially desirable.

As a method of regenerating a titanosilicate catalyst which has been used in epoxidation reaction of propylene, for example, a method of treating TS-1, which has been used in the epoxidation reaction of propylene with hydrogen peroxide, at 385° C. in the presence of nitrogen gas has been reported (see EP0790075B1). However, a method of calcining at a high temperature requires a special apparatus, and needs steps of taking out and drying a catalyst and other steps and accordingly it is industrially undesirable. Furthermore, it has been found that there arises a new problem that not a little amount of propylene glycol is by-produced when epoxidation reaction of propylene is actually performed with a titanosilicate catalyst which has been treated by this method and using oxygen and hydrogen in substitution for hydrogen peroxide.

### DISCLOSURE OF THE INVENTION

The present invention relates to a method suitable for regeneration of titanosilicate catalysts which enables to readily regenerate the catalytic activity of titanosilicate catal_{y}sts deteriorated in the catalytic ability without affecting the catalytic reaction by the solvent used for regeneration.

That is, the present invention relates to a regeneration method of a titanosilicate catalyst which comprises contacting the titanosilicate catalyst deteriorated in catalytic ability with a nitrile compound or a mixture of water and a nitrile compound at a temperature from 25° C. to 200° C. (hereinbelow referred to as the regenerating method of the present invention), and a method of producing propylene oxide which comprises reacting propylene, oxygen and hydrogen in a liquid phase comprising acetonitrile or a mixed solvent of acetonitrile and water in the presence of a titanosilicate catalyst regenerated by the method of the present invention and a noble metal catalyst (hereinbelow referred to as the production method of propylene oxide of the present invention).

According to the present invention, titanosilicate catalysts which have been used for reaction and deteriorated in the catalytic ability can be readily regenerated by using a nitrile compound or a mixture of water and a nitrile compound. In addition, the production reaction of propylene oxide using a titanosilicate catalyst regenerated by the method of the present invention has an advantage that by-production of propylene glycol is decreased. When a solvent containing acetonitrile is used in the reaction using a titanosilicate catalyst, there is no fear of incorporation of a solvent and such a method is advantageous as an industrial production method.

### BEST MODES FOR CARRYING OUT THE INVENTION

The regeneration method of a titanosilicate catalyst of the present invention is typically suitable for regeneration of titanosilicate which has been used as a catalyst in the method of producing propylene oxide which comprises reacting propylene, oxygen and hydrogen in a liquid phase comprising acetonitrile or a mixed solvent of acetonitrile and water in the presence of a noble metal catalyst and deteriorated in the catalytic ability, and usually those deteriorated in the catalytic ability are separated from the reaction system and treated by the regenerating method.

At first, titanosilicate used in the production method of propylene oxide as a catalyst is described. It is a general term representing an entity in which a part of Si of a porous silicate (SiO₂) is replaced with Ti. Ti of titanosilicate is within the SiO₂ skeleton, and the fact that Ti is contained within the SiO₂ framework can be easily confirmed by the presence of a peak at 210 nm to 230 nm in ultraviolet to visible absorption spectrum. In addition, Ti of TiO₂ is usually 6-coordinate but Ti of titanosilicate is 4-coordinate, and therefore it can be confirmed easily by measuring a coordination number by titanium K-edge XAFS analysis, etc.

Specific examples of titanosilicate include crystalline titanosilicate such as TS-1 having MFI structure, TS-2 having MEL structure, Ti-ZSM-12 having MTW structure (for example, those described in Zeolites 15, 236-242, (1995)), Ti-Beta having BEA structure (for example, those described in Journal of Catalysis 199, 41-47, (2001)), Ti-MWW having MWW structure (for example, those described in Chemistry Letters 774-775, (2000)), Ti-UTD-1 having DON structure (for example, those described in Zeolites 15, 519-525, (1995)) according to the structural code of IZA (International Zeolite Association). In addition to these, examples of layered titanosilicate include Ti-MWW precursor (for example, those described in Japanese Laid-Open Patent Publication No. 2003-32745) and Ti-YNU-1(for example, those described in Angewandte Chemie International Edition 43, 236-240, (2004).

Of these titanosilicates, crystalline titanosilicates or layered titanosilicates having fine pores of oxygen 12-membered ring or larger are preferable judging from the productivity of propylene oxide. The crystalline titanosilicates having fine pores of oxygen 12-membered ring or larger include Ti-ZSM-12, Ti-Beta, Ti-MWW and Ti-UTD-1. The layered titanosilicates having fine pores of oxygen 12-membered ring or larger include Ti-MWW precursor and Ti-YNU-1. More preferable titanosilicates include Ti-MWW and Ti-MWW precursor.

A Ti-MWW precursor is commonly synthesized by a method comprising contacting a layered compound directly hydrothermally synthesized from a boron compound, a titanium compound, a silicon compound and a structure directing agent, which layered compound is also referred to as a “as-synthesized sample”, with a strong acid aqueous solution under refluxing condition to thereby remove the structure directing agent so that the molar ratio of silicon to nitrogen (Si/N ratio) may be adjusted to be 21 or more (see, for example, Japanese Laid-Open Patent Publication No. 2005-262164). In the meantime, Catalysis Today, 117 (2006), 199-205 discloses that a Ti-MWW precursor containing 13.5 to 14.2 wt % of nitrogen can be obtained by subjecting a compound obtained by mixing Ti-MWW, piperidine and water to hydrothermal treatment followed by washing with water. The Si/N ratio of this Ti-MWW precursor is calculated to be 8.5 to 8.6 from the results of CHN elemental analysis, Si/Ti ratio and Si/B ratio described in the above document, which is higher in nitrogen content compared to the conventionally known Ti-MWW precursors but this Ti-MWW precursor can be used as a preferable titanosilicate (Ti-MWW precursor). Ti-MWW can be obtained by crystallizing the thus obtained Ti-MWW precursor by calcining.

The titanosilicate includes, for example, those silylated with a silylating agent such as 1,1,1,3,3,3-hexamethyldisilazane. Silylated titanosilicate is also preferable titanosilicate (for example, silylated Ti-MWW, etc.) since the activity or selectivity can be further enhanced by silylation.

The method of the present invention is effective for recovering the activity of titanosilicate which has been used for reaction and deteriorated in the catalytic ability (hereinbelow also referred to as deteriorated titanosilicate). The method has also excellent characteristics that by-production of the propylene glycol is caused less than a conventionally known method of regenerating deteriorated titanosilicate comprising treatment at a high temperature in the presence of a suitable gas.

The method of the present invention is described in the following by way of a reaction to produce propylene oxide as an example. In this reaction process, when a method of generating hydrogen peroxide from hydrogen and oxygen in the reaction system and oxidizing propylene is adopted, a noble metal catalyst is used. The constituent elements of such a noble metal catalyst include palladium, platinum, ruthenium, rhodium, iridium, osmium, gold or an alloy or a mixture thereof. Preferable noble metals include palladium, platinum and gold. More preferable noble metal is palladium. Palladium can be used by adding and mixing with a metal such as platinum, gold, rhodium, iridium and osmium. Preferable metal to be added includes platinum. These noble metals may be in the state of a compound such as an oxide or a hydroxide. That is, they may be filled in a reactor in the state of a noble metal compound and partially or wholly reduced by hydrogen in the reaction raw materials under the reaction conditions and used.

The noble metals are usually used while supported by a carrier. The noble metal may be supported by titanosilicate for use or supported for use on a carrier other than titanosilicate, that is, oxides such as silica, alumina, titania, zirconia and niobia, hydrates such as niobic acid, zirconic acid, tungstic acid, titanic acid or carbon and mixtures thereof. When a noble metal is supported by a carrier other than titanosilicate, the carrier having a noble metal supported thereon may be mixed with titanosilicate and the mixture may be used as a catalyst. Among the carrier other than titanosilicate, carbon can be included as a preferable carrier. Examples of the carbon carrier include activated carbon, carbon black, graphite and carbon nanotube.

As a method for preparing a noble metal supported catalyst, a method comprising supporting an ammine complex such as Pd tetraammine chloride on a carrier by impregnation method and the like and then performing reduction. As for a reduction method, reduction may be performed with a reducing agent such as hydrogen or reduction may be performed with ammonia gas generated at the time of thermolysis under an inert gas. The reduction temperature varies depending on the noble metal ammine complex but when Pd tetraammine chloride is used, it is typically from 100° C. to 500° C., and preferably from 200° C. to 350° C. The thus obtained noble metal supported carrier contains the noble metal typically in the range of 0.01 to 20% by weight, and more preferably 0.1 to 5% by weight. The weight ratio of the noble metal to titanosilicate (used for the reaction (weight of noble metal/weight of titanosilicate) is preferably 0.01 to 100% by weight, and more preferably 0.1 to 20% by weight. The reaction to produce propylene oxides is usually performed in a liquid phase consisting of a mixed solvent of a nitrile compound and water. Preferable nitrile compounds include linear or branched chain saturated aliphatic nitriles or aromatic nitriles. Examples of these nitrile compounds include C₂ to C₄ alkylnitriles such as acetonitrile, propionitrile, isobutyronitrile, butyronitrile and benzonitrile, and acetonitrile is preferable.

The ratio of water to the nitrile compound is usually 90:10 to 0.01:99.99 by weight ratio, and preferably 50:50 to 0.01:99.99. When the ratio of water becomes excessive, there is a case that propylene oxide tends to react with water and deteriorate by ring cleavage, and there is also a case that the selectivity of propylene oxide lowers. On the contrary, collection cost of the solvent increases when the ratio of the nitrile compound becomes excessive.

In the reaction to produce propylene oxides, the method to add a buffer salt to the reaction solvent is effective since it prevents decrease in the catalytic activity and further increases catalytic activity and enables to improve use efficiency of hydrogen. The buffer salt may be used with a noble metal or the buffer salt and the noble metal may be used independently. The added amount of the buffer salt is usually 0.001 mmol/kg to 100 mmol/kg per unit solvent weight (total weight of water and organic solvent). Examples of the buffer salt include buffer salts which consist of 1) an anion selected from a sulfate ion, a hydrogen sulfate ion, a carbonate ion, a hydrogen carbonate ion, a phosphate ion, a hydrogen phosphate ion, a dihydrogen phosphate ion, a hydrogen pyrophosphate ion, a pyrophosphate ion, a halogen ion, a nitrate ion, a hydroxyl ion or a C₁ to C₁₀ carboxylate ion and 2) a cation selected from ammonium, alkylammonium, alkylaryl ammonium, alkali metal or alkaline earth metal. Examples of the C₁ to C₁₀ carboxylate ion include an acetate ion, a formic acid ion, a propionate ion, a butyric acid ion, a valeric acid ion, a caproic acid ion, a caprylic acid ion, a capric acid ion and a benzoate ion. Examples of the alkyl ammonium include tetramethylammonium, tetraethylammonium, tetra-n-propylammonium, tetra-n-butylammonium, cetyltrimethylammonium. Examples of the alkali metal or alkaline-earth metal cations include a lithium cation, a sodium cation, a potassium cation, a rubidium cation, a cesium cation, a magnesium cation, a calcium cation, a strontium cation and a barium cation. Examples of a preferable buffer salt include ammonium salts of an inorganic acid such as ammonium sulfate, ammonium hydrogen sulfate, ammonium carbonate, ammonium hydrogen carbonate, diammonium hydrogen phosphate, ammonium dihydrogen phosphate, ammonium phosphate, hydrogen pyrophosphate ammonium, pyrophosphate ammonium, ammonium chloride and ammonium nitrate or ammonium salts of a C₁ to C₁₀ carboxylic acid such as ammonium acetate, and diammonium hydrogen phosphate is preferable as a ammonium salt.

Examples of a production reaction of propylene oxide include fixed bed flow reaction and a complete mixing slurry flow reaction. The partial pressure ratio of oxygen and hydrogen to supply to the reaction vessel is typically in the range of 1:50 to 50:1. The partial pressure ratio of oxygen and hydrogen is preferably 1:2 to 10:1. When the partial pressure ratio of oxygen and hydrogen (oxygen/hydrogen) is too high, there is a case that generation rate of propylene oxide lowers. In the meantime, when the partial pressure ratio of oxygen and hydrogen (oxygen/hydrogen) is too low, there is a case that the selectivity of propylene oxide decreases due to the increase in the propane by-production. Oxygen and hydrogen gas usable in this reaction can be diluted with a gas for dilution and subjected to the reaction. Examples of the gas for dilution include nitrogen, argon, carbon dioxide, methane, ethane and propane. The concentration of the gas for dilution is not limited in particular but oxygen or hydrogen is diluted as needed and subjected to the reaction. The oxygen includes molecular oxygen such as an oxygen gas or air. As an oxygen gas, an oxygen gas produced by inexpensive pressure swing method can be used and a high purity oxygen gas produced by low temperature separation can be used as required. The reaction temperature in the production reaction of propylene oxide is typically 0° C. to 150° C., and preferably 40° C. to 90° C. The reaction pressure is not limited in particular but it is typically 0.1 MPa to 20 MPa in gauge pressure, and preferably 1 MPa to 10 MPa. Collection of propylene oxide which is the product of the reaction can be performed by normal distillation separation.

The deteriorated titanosilicate which has been used for such a production reaction of propylene oxide and deteriorated in the catalytic ability is separated from the reaction system and it may be subjected to the regeneration treatment as in the form used for the reaction or may be subjected to the regeneration process after crushing the taken out catalyst as needed. There are no problems for subjecting the deteriorated titanosilicate to the regeneration process even when the deteriorated titanosilicate is in a mixed state with a catalyst other than the deteriorated titanosilicate used in the reaction and a reaction fillers such as alumina.

In the method of the present invention, the titanosilicate catalyst which has been deteriorated in the catalytic ability is separated from the reaction system and be subjected to the regeneration process. The separation from the reaction system is performed in consideration of the catalyst, the reaction method and the shape of the reactor used. For example, it is performed by a method of physically taking out the catalyst from the reactor in which the catalytic reaction is proceeding, or by controlling the flow of any of the reaction gas of hydrogen, oxygen and propylene (hereinbelow referred to as reaction gas) which is the reaction agent in the reaction system in the reactor or the packed column in which the titanosilicate catalyst is contained, or by separating the flow of the gas in a part of the reactor or the packed column by switching the flow path of the reaction gas from the reactor and the like thereby controlling the flow of the gas in said part of the reactor or the packed column. At this time, the operation is preferably performed while the generation reaction of propylene oxide is stopped. The operation to control the flow may be performed by stopping the supply of at least one kind of the reaction gas or replacing a part of or whole of the reaction gas with an inert gas.

As for the nitrile compound or the mixture of water and a nitrile compound used for regeneration of the deteriorated titanosilicate separated from reaction system, those collected from the solvent used in the production process of propylene oxide may be used and the raw material propylene remaining in the reaction system in the production process of propylene oxide, generated hydrogen peroxide or propylene oxide may be allowed to remain as long as the effect of the present invention is not impaired. The ratio of water and a nitrile compound usually used for the case that the regeneration of the deteriorated titanosilicate catalyst is performed with a nitrile compound or a mixture of water and a nitrile compound is 90:10 to 0:100 by the weight ratio, preferably 80:20 to 0:100, more preferably 50:50 to 0:100.

The contact of the deteriorated titanosilicate catalyst and a nitrile compound or mixture of water and a nitrile compound is performed in a temperature range from 25° C. to 200° C. but it is more preferably performed in a temperature range from 65° C. to 130° C., and particularly preferably performed in a temperature range from 65° C. to 80° C. There is no limitation in particular on the pressure at the time of the contact but it is usually treated at 0 to 10 MPa in gauge pressure.

The step of contacting the deteriorated titanosilicate catalyst separated from the reaction system with a nitrile compound or mixture of water and a nitrile compound is usually carried out by flow method or batch method. It is also possible to supply gas into a liquid for the purpose of improving dispersion of the catalyst in the liquid. The gas ingredients of the atmosphere at this time is not limited in particular and hydrogen and oxygen used for reaction may remain, and it may be an inert gas atmospheres such as nitrogen. Regenerated titanosilicate can be obtained from the deteriorated titanosilicate (hereinafter referred to as the regeneration titanosilicate). The regeneration titanosilicate can be used in the reaction to produce propylene oxides by reacting propylene, oxygen and hydrogen along with a noble metal catalyst.

The method to separate from the reaction system the titanosilicate catalyst which has been deteriorated in the catalytic ability in a reaction comprising reacting propylene, hydrogen, oxygen in a liquid phase and reacting propylene to produce propylene oxide and the regeneration process are specifically described by way of the reaction mode and the shape of the reactor, for an example.

In the case of a fixed bed type reactor characterized by filling up a tower type container with a catalyst or filling up the inside or the outside of a multitube heat exchanger with a catalyst and supplying a nitrile compound or a mixture of water and a nitrile compound, propylene, hydrogen, oxygen, nitrogen gases from the lower part of the reactor, and performing reaction in a state that a part or whole of the filled catalyst remains in the tower type container, or in the case of a suspension tank reactor characterized by imparting gas, liquid and/or solid with kinetic energy with a rotating agitating blade or a pump to fluidize and mix the gas, liquid and a catalyst, the titanosilicate catalyst is maintained in the state that the catalyst stays in the reactor after the reaction is terminated, and the titanosilicate catalyst can be regenerated by contacting a nitrile compound or a mixture of water and a nitrile compound while keeping the catalyst layer in a suitable temperature range of 25 to 200° C.

In order to terminate the production reaction of propylene oxide, it is preferable from the aspect of safety and prevention of accidents to stop supplying propylene after the supply of oxygen and hydrogen is terminated and subsequently stop supplying a nitrile compound or a mixture of water and a nitrile compound and nitrogen in this order. It is preferable to take out the reaction liquid which remains in the reactor after having stopped the reaction and to substitute the void spaces among the catalyst particles with an inert gas for the purpose of enhancing cleaning effect by a nitrile compound or a mixture of water and a nitrile compound described later but the regeneration treatment with a nitrile compound or a mixture of water and a nitrile compound may be performed in the state that the reaction liquid remains within the reactor.

The regeneration of the deteriorated titanosilicate catalyst can be performed, for example, by allowing the titanosilicate catalyst to stay in the reactor and to contact with a nitrile compound or a mixture of water and a nitrile compound in the state that the temperature of the fixed bed or of the catalyst layer of the fluid bed in the reactor is maintained at a suitable temperature within 25 to 200° C. More particularly, for the contact of the catalyst layer and a nitrile compound or a mixture of water and a nitrile compound, a method of continuously supplying and discharging a nitrile compound or a mixture of water and a nitrile compound and a method of performing one or more cycles of supplying a nitrile compound or a mixture of water and a nitrile compound in a volume of a part of or whole of the void spaces in the catalyst layer or in a volume larger than the void spaces in the catalyst layer and taking out the same after immersing the catalyst layer for a predetermined time, and a method of combining these can be included. The latter is suitable from a viewpoint of reducing the amount of a nitrile compound or a mixture of water and a nitrile compound to be used. The pressure at which the regeneration is performed is the atmospheric pressure or the saturated vapor pressure of the nitrile compound or the mixture of water and a nitrile compound at the temperature of performing regeneration or a pressure higher than these. The amount of the nitrile compound or the mixture of water and a nitrile compound to be used for regeneration is one or more times of the catalyst weight, preferably 10 or more times thereof, more preferably 100 or more times thereof.

The reaction liquid take out after the reaction is stopped may be removed or recycled to the reaction process after it is subjected to purification treatment such as filtration or distillation and then propylene oxide, propylene, the nitrile compound or the mixture of water and a nitrile compound are collected or may be discarded without being subjected to purification treatment. The nitrile compound or the mixture of water and a nitrile compound contacted with a deteriorated titanosilicate catalyst may be discarded as it is, or subjected to regeneration treatment without purification treatment but it is preferably used as a solvent for the reaction after subjected to distillation, adsorption, filtration to remove impurities.

The nitrile compound or the mixture of water and a nitrile compound remaining in the reactor after contacted with a titanosilicate catalyst may be discarded as it is, or subjected to regeneration treatment without purification treatment or may be used as a solvent for the reaction after subjected to distillation, adsorption, filtration to remove impurities but it is desirably in the remaining state and supplied with hydrogen, oxygen, propylene, which are reaction raw materials, and a nitrile compound or a mixture of water and a nitrile compound and nitrogen.

The regeneration method of a catalyst of the present invention is performed under predetermined temperature conditions, and the temperature adjustment is preferably performed using temperature adjustment means such as a multitube heat exchanger, a plate type heat exchanger, a spiral type heat exchanger with heat medium such as steam, heat transfer oil, molten salt and water. In order to adjust temperature, a nitrile compound or a mixture of water and a nitrile compound the temperature of which is adjusted beforehand may be supplied, or the temperature may be adjusted in the reactor. It is sufficient to be provided with heating or cooling function. As an equipment for adjusting the temperature, it is desirably provided with a measuring apparatus which can measure the temperature of the nitrile compound or the mixture of water and a nitrile compound the temperature of which is adjusted and an equipment which can regulate the flow rate and temperature of the heat medium so that the measured temperature may be the predetermined temperature.

It is desirable that a flowmeter to measure the flow rate of a nitrile compound or a mixture of water and a nitrile compound and a mechanism to adjust the flow rate, preferably a control valve and a measuring instrument which can calculate the integrated flow amount are disposed in the inlet of such a reactor.

It is desirable that the reactor has a volume sufficient for securing detention time suitable for the reaction and ability of enduring the temperature and pressure, and has a pressure gauge for measuring pressure and a thermometer for measuring temperature, and in the case of fixed bed type reactor, it is equipped with wire netting, filter cloth, sintered metal or the like on the top and the bottom of the catalyst layer to prevent outflow of the catalyst and has a heat exchange function for the purpose of regulating the temperature. It is sufficient that it has a function to heat exchange with heat medium such as steam, heat transfer oil, molten salt and water and in the case of a suspension layer type reactor, the reactor has an agitating blade for imparting gas, liquid and solid with energy and a pump in the inside or outside of the reactor and wire netting, filter cloth, sintered metal or the like is disposed within the reactor or the piping connected to the reactor to prevent outflow of the titanosilicate catalyst. In the fixed bed type reactor, it is desirably provided with a dispersion board to scatter raw materials or a sparger ring for the purpose of scattering gas at the lower part of the reactor.

When the reaction apparatus used for the production of propylene oxide is a reaction apparatus consisting of plural reactors, a nitrile compound or a mixture of water and a nitrile compound may be supplied in a condition that one or more reactors are connected in series or in parallel, or the regeneration procedure may be conducted respectively. While one or more reactors perform regeneration process, the reaction may be stopped in the other reactors by stopping the supply of raw materials, but it is preferable to continue the reaction from a viewpoint of economy.

When a noble metal catalyst and a titanosilicate catalyst are filled up independently in respective reactors and the regeneration treatment can be performed individually, the regeneration treatment may be performed each individually or regeneration may be simultaneously performed by supplying a nitrile compound or a mixture of water and a nitrile compound to the reactors connected in series or in parallel.

The catalyst present in the reactor which is not yet regenerated may be just taken out as it is or a part thereof or the whole thereof may be taken out of the reactor, or the catalyst may be regenerated after adding a new catalyst or a separately regenerated catalyst equivalent to the taken out portion in the dry weight. The catalyst the regeneration of which has been completed in the reactor may be just used as it is in the whole amount or a part thereof or the whole thereof may be taken out of the reactor while adding a new catalyst or a separately regenerated catalyst equivalent to the taken out portion in the dry weight.

In the case of a suspension tank reactor characterized by imparting gas, liquid and solid with kinetic energy by an agitating blade or a pump so as to make flow and mix the gas, liquid and a catalyst, regeneration of the catalyst can be performed by transferring the catalyst from the reactor to the regeneration equipment while the reaction is performed or after the reaction is stopped by stopping the supply of the raw materials and contacting the catalyst with a nitrile compound or a mixture of water and a nitrile compound in such a condition that the temperature of the taken out titanosilicate catalyst may be adjusted to 25 to 200° C.

When the reaction is stopped for the regeneration of the catalyst, it is sufficient to stop the supply of raw materials for stopping the regeneration reaction, and it is preferable from the aspect of safety and prevention of accidents to stop supplying propylene after the supply of oxygen and hydrogen is terminated and subsequently stop supplying a nitrile compound or a mixture of water and a nitrile compound and nitrogen in this order. The regeneration treatment may be performed by taking out the titanosilicate catalyst and the reaction liquid which remain in the reactor after having stopped the reaction or by taking out the reaction liquid intermittently or continuously while the reaction is allowed to continue within the reactor.

The regeneration of the catalyst may be performed without stopping the production reaction of propylene oxide by intermittently or continuously taking out a part of the catalyst and the reaction liquid in the reactor by way of a nozzle disposed in the reactor making use of gravity, difference in pressure and subjecting it to the process of regenerating a catalyst.

The taken out titanosilicate catalyst and reaction liquid may be transferred to a regenerating equipment having a solid-liquid separating function and subjected to solid-liquid separation in a solid-liquid separating apparatus such as a centrifuging type filter, a leaf filter, a filter press and a superdecanter or preferably in an agitation tank having a solid-liquid separating function represented by an agitation tank having a candle filter.

When the regeneration is performed by a solid-liquid separating equipment, the catalyst and the reaction liquid taken out of the reactor is subjected to solid-liquid separation, and preferably after removing the remaining mother liquid by subjecting the filtered wet catalyst to pressurizing compress, centrifugal force or the like, a nitrile compound or a mixture of water and a nitrile compound adjusted to 20 to 200° C. is intermittently or continuously supplied to the wet catalyst in amount of one or more times, preferably 10 or more times, more preferably 100 or more times of the dry weight of the wet catalyst, and thereby the catalyst can be contacted with the nitrile compound or the mixture of water and a nitrile compound and regenerated. It is sufficient that the solid-liquid separation has a separating function by the physical size of the particles such as wire netting, sintered metal and filter cloth. A part of or the whole of the regenerated wet catalyst is intermittently or continuously taken out to the agitation tank as wet powder from a filter, and mixed with a nitrile compound or a mixture of water and a nitrile compound in the agitation tank to become a slurry, and is recycled to the reactor. The part of the whole of the regenerated wet catalyst is directly recycled to the reactor, or discarded or regenerated by the other method. In order to supplement the reaction apparatus with a catalyst not recycled, a new catalyst or a separately regenerated catalyst equivalent to the portion taken out of the system in the dry weight may be added. It is sufficient that the agitation tank for making a slurry has a function of imparting solid and liquid with kinetic energy and performing mixing, and performing complete mixing is not required. It is sufficient that the rate of supplying the nitrile compound or the mixture of water and the nitrile compound to the wet catalyst is a rate which enables to keep a state that the wet powder surface is covered with the nitrile compound or the mixture of water and a nitrile compound in the regeneration operation.

When the regeneration is performed by an agitation tank having a solid-liquid separating function, the catalyst and the reaction liquid taken out of the reactor is extracted to the agitation tank the temperature of which is adjusted to 20 to 200° C., a nitrile compound or a mixture of water and a nitrile compound, which is selected to be in amount of one or more times, advantageously 10 or more times, more advantageously 100 or more times of the dry weight of the wet catalyst, is intermittently or continuously supplied to the agitation tank, and intermittently or continuously extracted via a solid-liquid separation equipment, thereby the catalyst can be contacted with the nitrile compound or the mixture of water and a nitrile compound and regenerated. The solid-liquid separation equipment may be in the inside of the agitation tank or disposed outside and filtering may be performed by circulation. It is sufficient that the solid-liquid separation has a separating function by the physical size of the particles such as wire netting, sintered metal and filter cloth. It is desirable that the agitation tank is equipped with an instrument for measuring the volume of liquid, a pressure gauge for measuring pressure and a thermometer for measuring temperature, and has a heat exchange function for the purpose of regulating the temperature. It is sufficient that it has a function to heat exchange with heat medium such as steam, heat transfer oil, molten salt and water and desirably has an agitating blade for imparting gas, liquid and solid with energy and a pump in the inside or outside of the reactor. A part of or the whole of the regenerated catalyst and a nitrile compound or a mixture of water and a nitrile compound is intermittently or continuously recycled to the reactor. In order to supplement the reaction apparatus with a catalyst not recycled, a new catalyst or a separately regenerated catalyst equivalent to the portion taken out of the system in the dry weight may be added. The part of the whole of the rest of the catalyst is discarded or regenerated by the other method. It is desirable that the agitation tank has a mechanism to enable to freely change the existence ratio of solid and liquid in the agitation tank, and from a viewpoint of reducing a nitrile compound or a mixture of water and a nitrile compound, the lower the existence ratio of liquid to solid, the more desirable.

The reaction liquid taken out after the reaction is stopped may be removed after it is subjected to purification treatment such as filtration or distillation and then valuable ingredients, that is, propylene oxide, propylene, the nitrile compound or the mixture of water and a nitrile compound are collected, or may be recycle to the reaction process or may be discarded without being subjected to purification treatment. The nitrile compound or the mixture of water and a nitrile compound which has been used for washing may be just discarded as it is or may be subjected to regeneration treatment in an untreated condition but it is desirable to use as a solvent for the reaction after subjected to distillation, adsorption, filtration to remove impurities.

As for means to adjust the temperature, it is preferable that multitube heat exchanger, plate type heat exchanger, spiral type heat exchanger or the like is used and the temperature is adjusted by heat medium such as steam, heat transfer oil, amolten salt and water. In order to adjust the temperature, a nitrile compound or a mixture of water and a nitrile compound the temperature of which is adjusted beforehand may be supplied, or the temperature may be adjusted in the solid-liquid separation apparatus or the agitation tank and it is sufficient to have heating or cool function. As an equipment for adjusting the temperature, it is desirably provided with a measuring apparatus which can measure the temperature of the nitrile compound or the mixture of water and a nitrile compound the temperature of which is adjusted and an equipment which can regulate the flow rate and temperature of the heat medium so that the measured temperature may be the predetermined temperature.

It is desirable that a flowmeter to measure the flow rate of a nitrile compound or a mixture of water and a nitrile compound and a mechanism to adjust the flow rate thereof, preferably a control valve and a measuring instrument which can calculate the integrated flow amount are disposed in the feeding inlet of the nitrile compound or the mixture of water and a nitrile compound to the solid-liquid separation apparatus or the agitation tank.

In the case of the reaction apparatus consisting of plural reactors, the catalyst and the reaction liquid can be intermittently or continuously taken out of one or more reactors and the regeneration treatment can be performed using one or more regeneration equipments.

In the case of a fixed bed type reactor characterized by filling up a tower type container with a catalyst or filling up the inside or the outside of a multitube heat exchanger with a catalyst and supplying a nitrile compound or a mixture of water and a nitrile compound, propylene, hydrogen, oxygen, nitrogen gases from the lower part of the reactor, and performing reaction in a state that a part or whole of the filled catalyst remains in the tower type container, the catalyst is transferred from the reactor to the regeneration apparatus after the reaction is stopped by stopping the raw materials supply, and the catalytic activity can be improved by contacting with a nitrile compound or a mixture of water and a nitrile compound while adjusting the temperature of the taken out catalyst to 25 to 200° C.

In order to terminate the reaction and regenerate the catalyst, it is sufficient to stop the supply of the raw materials, and it is preferable from the aspect of safety and prevention of accidents to stop supplying propylene after the supply of oxygen and hydrogen is terminated and subsequently stop supplying a nitrile compound or a mixture of water and a nitrile compound and nitrogen in this order. It is preferable to take out the reaction liquid which remains in the reactor after having stopped the reaction and to substitute the void spaces among the catalyst particles with an inert gas but it is more preferable from a viewpoint of removing the remaining non-volatile components to supply a nitrile compound or a mixture of water and a nitrile compound in the state that the reaction liquid remains within the reactor, replace the same with the nitrile compound or the mixture of water and a nitrile compound and then take out the remaining liquid.

It is desirable from the viewpoint of safety and prevention of accidents to remove a part of or the whole of the volatile components which remain in the reactor by drawing to vacuum while heating the reactor or supplying an inert gas like heated nitrogen since such operation lowers the possibility of inflammation of the inflammable materials when the catalyst is taken out.

As another method of taking out catalyst in the reactor, a method can be included which comprises taking out a mixture of solid and liquid while supplying a nitrile compound or a mixture of water and a nitrile compound to the reactor after having stopping the reaction, performing solid-liquid separation with a solid-liquid separation apparatus and removing a part of or the whole of the remaining volatile components in the solid-liquid separation apparatus or with a dryer.

The reaction liquid taken out after the reaction is stopped may be removed after it is subjected to purification treatment such as filtration or distillation and then valuable ingredients, that is, propylene oxide, propylene, the nitrile compound or the mixture of water and a nitrile compound are collected, or may be recycle to the reaction process or may be discarded without being subjected to purification treatment. The nitrile compound or the mixture of water and a nitrile compound which has been used for substituting the reaction liquid is desirably used as a solvent for the reaction after subjected to distillation, adsorption, filtration to remove impurities. It may be just discarded as it is or may be subjected to regeneration treatment in an untreated condition.

The catalyst in the reactor or in the solid-liquid separation apparatus or in the dryer can be taken out by gravity from the aperture provided in the lower part. When the catalyst does not flow by itself and cannot be discharged, a part of or the whole of the catalyst may be fluidized and discharged using gas such as air or nitrogen. Alternatively, a pipe is put in the reactor and the catalyst can be taken out and transferred with air flow by supplying gas such as air or nitrogen in the reactor while drawing the inside of the pipe at low pressure. The catalyst which has been taken out of the reactor may be just transferred to the regeneration equipment or may be transferred to the regeneration equipment after having filled it up in the other container. The part of or the whole of the taken out catalyst may be discarded or may be regenerated by another method.

The taken out catalyst and reaction liquid is transferred to the regeneration equipment having solid-liquid separation function. The equipment to perform solid-liquid separation may be a solid-liquid separating apparatus such as a centrifuging type filter, a leaf filter, a filter press and a superdecanter but it may be an agitation tank having a solid-liquid separating function represented by an agitation tank having a candle filter.

When the regeneration is performed by a solid-liquid separating equipment, the catalyst and the reaction liquid taken out of the reactor is subjected to solid-liquid separation, and preferably after removing the remaining mother liquid by subjecting the filtered wet catalyst to pressurizing compress, centrifugal force or the like, a nitrile compound or a mixture of water and a nitrile compound adjusted to 20 to 200° C., which is selected to be in amount of one or more times, advantageously 10 or more times, more advantageously 100 or more times of the dry weight of the wet catalyst, is intermittently or continuously supplied to the wet catalyst and thereby the catalyst can be contacted with the nitrile compound or the mixture of water and a nitrile compound and regenerated. It is sufficient that the solid-liquid separation has a separating function by the physical size of the particles such as wire netting, sintered metal and filter cloth. A part of or the whole of the regenerated wet catalyst is intermittently or continuously taken out as wet catalyst from a filter. The part of or the whole of the regenerated wet catalyst is directly recycled to the reactor, or discarded or regenerated by the other method. In order to supplement the reaction apparatus with a catalyst not recycled, a new catalyst or a separately regenerated catalyst equivalent to the portion taken out of the system in the dry weight may be added. It is sufficient that the agitation tank for making a slurry has a function of imparting solid and liquid with kinetic energy and performing mixing, and performing complete mixing is not required.

When the regeneration is performed by an agitation tank having a solid-liquid separating function, the catalyst taken out of the reactor is extracted to the agitation tank the temperature of which is adjusted to 20 to 200° C., a nitrile compound or a mixture of water and a nitrile compound, which is selected to be in amount of one or more times, advantageously 10 or more times, more advantageously 100 or more times of the dry weight of the wet catalyst, is intermittently or continuously supplied to the agitation tank, and intermittently or continuously extracted via a solid-liquid separation equipment, and thereby the catalyst can be contacted with the nitrile compound or the mixture of water and a nitrile compound and regenerated. The solid-liquid separation equipment may be in the inside of the agitation tank or disposed outside and filtering may be performed by circulation. It is sufficient that the solid-liquid separation has a separating function by the physical size of the particles such as wire netting, sintered metal and filter cloth. It is desirable that the agitation tank is equipped with an instrument for measuring the volume of liquid, a pressure gauge for measuring pressure and a thermometer for measuring temperature, and has a heat exchange function for the purpose of regulating the temperature. It is sufficient that it has a function to heat exchange with heat medium such as steam, heat transfer oil, molten salt and water and desirably has an agitating blade for imparting gas, liquid and solid with energy and a pump in the inside or outside of the reactor. It is desirable that the agitation tank has a mechanism to enable to freely change the existence ratio of solid and liquid in the agitation tank, and from a viewpoint of reducing a nitrile compound or a mixture of water and a nitrile compound, the lower the existence ratio of liquid to solid, the more desirable. The regenerated wet catalyst is subjected to solid-liquid separation and part or the whole thereof is continuously or intermittently taken out the as a wet catalyst. The part of the whole of the regenerated wet catalyst is directly recycled to the reactor, or discarded or regenerated by the other method. In order to supplement the reaction apparatus with a decreased portion of the catalyst not recycled, a new catalyst or a separately regenerated catalyst equivalent to the portion taken out of the system in the dry weight may be added.

When the wet catalyst is recycled to the reactor or discarded or subjected to regeneration by another method, the catalyst may catch fire by nitril vapor if it contacts with air, and accordingly, it is preferably to be taken out after dried. The drying removes a part of or the whole of the volatile components which remain in the solid-liquid separation equipment by drawing the solid-liquid separation to vacuum while heating the same or by supplying a heated inert gas like nitrogen.

As means to adjust the temperature, it is preferable to use a multitube heat exchanger, a plate type heat exchanger, a spiral type heat exchanger with heat medium such as steam, heat transfer oil, molten salt, water or the like. In order to adjust the temperature, a nitrile compound or a mixture of water and a nitrile compound the temperature of which is adjusted beforehand may be supplied, or the temperature may be adjusted in the solid-liquid separation apparatus or the agitation tank and it is sufficient to have heating or cool function. As an equipment for adjusting the temperature, it is desirably provided with a measuring apparatus which can measure the temperature of the nitrile compound or the mixture of water and a nitrile compound the temperature of which is adjusted and an equipment which can regulate the flow rate and temperature of the heat medium so that the measured temperature may be the predetermined temperature.

It is desirable that a flowmeter to measure the flow rate of a nitrile compound or a mixture of water and a nitrile compound and a mechanism to adjust the flow rate thereof, preferably a control valve and a measuring instrument which can calculate the integrated flow amount are disposed in the feeding inlet of the nitrile compound or the mixture of water and a nitrile compound to the solid-liquid separation apparatus or the agitation tank.

In the case of the reaction apparatus consisting of plural reactors, the catalyst can be continuously taken out of one or more reactors and the regeneration treatment can be performed using one or more regeneration equipments.

### EXAMPLES

In the following, the present invention is described by way of Examples but the present invention is not limited to these Examples.

### Example 1

Ti-MWW was prepared as follows. That is, 32 g of TBOT (tetra-n-butylorthotitanate), 162 g of boric acid and 117 g of fumed silica (cab-o-sil M7D) were dissolved in 257 g of piperidine and 686 g of pure water at room temperature under Air atmosphere in an autoclave while stirring to prepare a gel, which was matured for 1.5 hours and hermetically closed. After the temperature was elevated for further eight hours while stirring, it was maintained at 165° C. for 120 hours to perform hydrothermal synthesis to obtain a suspension solution. The obtained suspension solution was filtered and then washed with water till the filtrate reached to the vicinity of pH10. Then the filtered lump was dried at 50° C. and a white powder which was still in a state containing water was obtained. 750 mL of 2N nitric acid was added to 15 g of the obtained powder and refluxed for 20 hours. Subsequently, the mixture was filtered, washed with water to around neutrality and sufficiently dried at 50° C. and to obtain 11 g of a white powder. As a result of measuring X-ray diffraction pattern of this white powder with an X-ray diffraction device using copper K-alpha radiation, it was confirmed to be a Ti-MWW precursor. The obtained Ti-MWW precursor was calcined at 530° C. for six hours and to obtain a Ti-MWW catalyst powder. It was confirmed by measuring an X-ray diffraction pattern that the obtained powder had MWW structure, and the titanium content by ICP emission analysis was 1.57% by weight.

Pd/activated carbon (AC) catalyst was prepared by the following method. 10 g of commercial AC (active carbon, powder, Lot:SDK3674, manufactured by Wako Pure Chemical Industries Co., Ltd.) was filled up a calcining pipe made of glass and hydrogen gas was flowed at 100 mL/min. The temperature was elevated to 300° C. for one hour and maintained at 300° C. for another hour. It was allow to cool to room temperature one hour later, and then taken out as hydrogen activation treatment activated carbon. 300 mL of an aqueous solution containing 0.30 mmol of Pd tetraammine chloride was prepared in a 500 mL eggplant flask. 3 g of the above hydrogen activation activated carbon was added to this aqueous solution and the solution was stirred for eight hours. After the stirring was terminated, moisture was removed with a rotary evaporator and further vacuum drying was performed at 80° C. for six hours. The obtained catalyst precursor powder was calcined under nitrogen atmosphere at 300° C. for six hours to obtain Pd/AC catalyst.

Deteriorated Ti-MWW was prepared by the following methods. That is, a gel consisting of 20 g of Ti-MWW prepared by the above method, 71 g of propylene oxide, 25 g of propylene glycol, 46 g of water and 183 g of acetonitrile was prepared in an autoclave at room temperature under Air atmosphere while stirring and the autoclave was hermetically closed. After the temperature was elevated for 30 minutes while further stirring, it was maintained at 60° C. for 6 hours to perform deterioration treatment to obtain a suspension solution. The obtained suspension solution was filtered and then washed with 2 L of water at 20° C. and 2 L of water/acetonitrile mixture (¼ weight ratio) at 20° C. Then the filtered lump was vacuum dried at 150° C. and 22 g of a deteriorated Ti-MWW powder was obtained. The titanium content by ICP emission analysis was 1.54% by weight.

The deterioration Ti-MWW prepared as above was regenerated by the following method. 2.5 g of the deterioration Ti-MWW, 40 g of water and 160 g of acetonitrile were added to a glass flask at room temperature under Air atmosphere and refluxed (oil bath temperature: 100° C., solution temperature: 77° C.) for 24 hours. Subsequently, the mixture was filtered and washed with 2 L of water and 2 L of water/acetonitrile mixture (¼ weight ratio). Furthermore, it was sufficiently dried in vacuum at 150° C. to obtain 2.3 g of powder. The titanium content by ICP emission analysis was 1.56% by weight.

An autoclave with a volume of 0.5 L in which the titanosilicate catalyst regenerated in this way was used as a reactor, and to this, raw materials gas having a volume ratio of propylene/oxygen/hydrogen/nitrogen=4/1/8/87 at a rate of 16 L/hour and a solution of water/acetonitrile=20/80 (weight ratio) at a rate of 108 mL/hour were supplied, and propylene oxides was produced by continuous reaction in the conditions: at a temperature of 60° C., at a pressure of 0.8 MPa (gauge pressure) and at a detention time of 90 minutes by taking out the reaction mixture from the reactor through a filter. The reaction mixture in the reactor at this period contains 161 g of the reaction solvent, 0.266 g of the regenerated Ti-MWW and 0.03 g of 1% by weight Pd/activated carbon. As a result of analyzing the liquid phase and the vapor phase taken out five hours later after the reaction started using gas chromatography, propylene oxide generation activity per unit Ti-MWW weight was 17.4 mmol-PO/g-Ti-MWW·h and propylene glycol selectivity (propylene glycol generation activity/(propylene glycol generation activity+propylene oxide generation activity)) was 1.9%.

### Example 2

The same experiment operation was performed as in the experiment of Example 1 except that the deteriorated catalyst was regenerated by being contacted with a water/acetonitrile mixture at a solution temperature of 60° C. instead of being regenerated by contact with a water/acetonitrile mixture at a reflux temperature. As a result of analyzing the liquid phase and the vapor phase taken out five hours later after the reaction started using gas chromatography, propylene oxide generation activity per unit Ti-MWW weight was 14.6 mmol-PO/g-Ti-MWW·h and propylene glycol selectivity was 2.0%.

### Referential Example 1

The same experiment operation was performed as in the experiment of Example 1 except that fresh Ti-MWW was used instead of using regenerated Ti-MWW. As a result of analyzing the liquid phase and the vapor phase taken out five hours later after the reaction started using gas chromatography, propylene oxide generation activity per unit Ti-MWW weight was 20.2 mmol-PO/g-Ti-MWW·h and propylene glycol selectivity was 3.8%.

### Referential Example 2

The same experiment operation was performed as in the experiment of Example 1 except that deteriorated Ti-MWW was used instead of using regenerated Ti-MWW. As a result of analyzing the liquid phase and the vapor phase taken out five hours later after the reaction started using gas chromatography, propylene oxide generation activity per unit Ti-MWW weight was 11.1 mmol-PO/g-Ti-MWW·h and propylene glycol selectivity was 1.7%.

### Comparative Example 1

The same experiment operation was performed as in the experiment of Example 1 except that the deteriorated catalyst was subjected to regeneration treatment with nitrogen gas at 350° C. for six hours instead of being regenerated by contact with a water/acetonitrile mixture at a reflux temperature. As a result of analyzing the liquid phase and the vapor phase taken out five hours later after the reaction started using gas chromatography, propylene oxide generation activity per unit Ti-MWW weight was 17.9 mmol-PO/g-Ti-MWW·h and propylene glycol selectivity was 2.5%.

### Comparative Example 2

The same experiment operation was performed as in the experiment of Example 1 except that the deteriorated catalyst was subjected to regeneration treatment with nitrogen gas at 530° C. for six hours instead of being regenerated by contact with a water/acetonitrile mixture at a reflux temperature. As a result of analyzing the liquid phase and the vapor phase taken out five hours later after the reaction started using gas chromatography, propylene oxide generation activity per unit Ti-MWW weight was 19.3 mmol-PO/g-Ti-MWVV·h and propylene glycol selectivity was 2.7%.

### Example 3

Ti-MWW precursor was prepared as follows. That is, 899 g of piperidine, 2402 g of pure water, 112 g of TBOT (tetra-n-butylorthotitanate), 565 g of boric acid and 410 g of fumed silica (cab-o-sil M7D) were stirred for dissolution at room temperature under Air atmosphere in an autoclave to prepare a gel, which was matured for 1.5 hours and then hermetically closed. After the temperature was elevated over eight hours while further stirring, it was maintained at 160° C. for 120 hours to perform hydrothermal synthesis to obtain a suspension solution. The obtained suspension solution was filtered and then washed with water till the filtrate reached to pH10.7. Then the filtered lump was dried at 50° C. till no further decrease in weight was observed to obtain 515 g of solid. 3750 mL of 2 M nitric acid was added to 75 g of the obtained solid and refluxed for 20 hours. Subsequently, the mixture was filtered, washed with water to around neutrality and vacuum dried at 150° C. till no further decrease in weight was observed to obtain 61 g of a white powder. As a result of measuring X-ray diffraction pattern and UV-visible absorption spectrum of this white powder, it was confirmed to be a Ti-MWW precursor. 60 g of the obtained white powder was calcined at 530° C. for six hours to obtain 54 g of a powder (Ti-MWW). It was confirmed by measuring an X-ray diffraction pattern and UV-visible absorption spectrum that the obtained powder was Ti-MWW. Further, the same operation as the above was performed twice, and 162 g of Ti-MWW in total was obtained.

300 g of piperidine, 600 g of pure water and 110 g of the Ti-MWW obtained as above were stirred for dissolution at room temperature under Air atmosphere in an autoclave to prepare a gel, which was matured for 1.5 hours and then hermetically closed. After the temperature was elevated over four hours while further stirring, it was maintained at 160° C. for 24 hours to perform hydrothermal synthesis to obtain a suspension solution. The obtained suspension solution was filtered and then washed with water till the filtrate reached to the vicinity of pH9. Then the filtered lump was dried in vacuum at 150° C. till further decrease in weight was not observed and 114 g of a white powder was obtained. As a result of measuring X-ray diffraction pattern of this white powder, it was confirmed to have an MWW precursor structure and the measurement results of UV-visible absorption spectrum revealed that it was titanosilicate. The titanium content by ICP emission analysis was 1.66% by mass.

Pd/activated carbon (AC) catalyst was prepared by the following method. 3 g of active carbon powder (manufactured by Wako Pure Chemical Industries Co., Ltd.) washed with 2 L of water beforehand and 300 mL of water were added to a 1 L eggplant flask and stirred in air at room temperature. To this suspension, 100 mL of an aqueous solution containing 0.30 mmol of Pd colloid (manufactured by Nikki Shokubai Kasei Co., Ltd.) was slowly added dropwise in air at room temperature. After the dropwise addition was completed, the suspension was further stirred in air at room temperature for eight hours. After the stirring ended, moisture was removed with a rotary evaporator and the residue was vacuum dried at 80° C. for six hours and furthermore calcined at 300° C. under nitrogen atmosphere for six hours to obtain a Pd/AC catalyst.

A mixture of a deteriorated Ti-MWW precursor and a Pd/AC catalyst was prepared by the following methods. A gel consisting of 4 g of Ti-MWW prepared by the above method, 0.3 g of Pd/AC catalyst, 71 g of propylene oxide, 25 g of propylene glycol, 46 g of water and 183 g of acetonitrile was prepared in an autoclave at room temperature under Air atmosphere while stirring and the autoclave was hermetically closed. After the temperature was elevated over one hour while further stirring, it was maintained at 90° C. for 6 hours to perform deterioration treatment to obtain a suspension solution. The obtained suspension solution was filtered and then washed with 2 L of water at 20° C. Then the filtered lump was vacuum dried at 150° C. and 3.7 g of a mixture power of a deteriorated Ti-MWW precursor and a Pd/AC catalyst was obtained.

The mixture of a deteriorated Ti-MWW precursor and a Pd/AC catalyst prepared as above was regenerated by the following method. 1.2 g of the mixture of a deteriorated Ti-MWW precursor and a Pd/AC catalyst, 17 g of water and 69 g of acetonitrile were added to a glass flask at room temperature under Air atmosphere and refluxed (oil bath temperature: 100° C., solution temperature: 77° C.) for one hour. Subsequently, the mixture was filtered and washed with 2 L of water. Furthermore, it was sufficiently dried in vacuum at 150° C. to obtain 1.1 g of powder.

An autoclave with a volume of 0.5 L and charged with 0.43 g of the mixture of a Ti-MWW precursor regenerated in this way and a Pd/AC catalyst was used as a reactor, and to this, raw materials gas having a volume ratio of propylene/oxygen/hydrogen/nitrogen=6.5/4.5/11/78 was supplied at a rate of 21.3 NL/hour and a solution of water/acetonitrile=20/80 (weight ratio) containing 0.7 mmol/kg of anthraquinone at a rate of 108 mL/hour, and propylene oxide was produced by continuous reaction in the conditions: at a temperature of 60° C., at a pressure of 0.8 MPa (gauge pressure) and at a detention time of 90 minutes by taking out the reaction mixture from the reactor through a filter. As a result of analyzing the liquid phase and the vapor phase taken, out five hours after the reaction started using gas chromatography, propylene oxide generation activity per unit Ti-MWW weight was 14.5 mmol-PO/g-Ti-MWW precursor·h and propylene glycol selectivity (propylene glycol generation activity/(propylene glycol generation activity+propylene oxide generation activity)) was 1.4%.

### Referential Example 3

The same experiment operation was performed as in the experiment of Example 3 except that 0.4 g of a fresh Ti-MWW precursor and 0.03 g of a fresh Pd/AC catalyst were used instead of 0.43 g of the mixture of a regenerated Ti-MWW precursor and a Pd/AC catalyst. As a result of analyzing the liquid phase and the vapor phase taken out five hours after the reaction started using gas chromatography, propylene oxide generation activity per unit Ti-MWW weight was 13.0 mmol-PO/g-Ti-MWW precursor·h and propylene glycol selectivity was 2.6%.

### Referential Example 4

The same experiment operation was performed as in the experiment of Example 3 except that a mixture of a deteriorated Ti-MWW precursor and a Pd/AC catalyst were used instead of the mixture of a regenerated Ti-MWW precursor and a Pd/AC catalyst. As a result of analyzing the liquid phase and the vapor phase taken out five hours after the reaction started using gas chromatography, propylene oxide generation activity per unit Ti-MWW precursor weight was 10.8 mmol-PO/g-Ti-MWW·h and propylene glycol selectivity was 1.5%.

1. A regeneration method of a titanosilicate catalyst which comprises contacting the titanosilicate catalyst deteriorated in catalytic ability with a nitrile compound or a mixture of water and a nitrile compound at a temperature from 25° C. to 200° C.

2. The method according to claim 1 wherein the titanosilicate is crystalline titanosilicate having fine pores of oxygen 12-membered ring or larger.

3. The method according to claim 1 wherein the titanosilicate is crystalline titanosilicate having MWW structure or a layered Ti-MWW precursor.

4. The method according to claim 1 wherein the nitrile compound is acetonitrile.

5. The method according to claim 1 wherein the temperature is from 65° C. to 130° C.

6. The method according to claim 1 wherein the temperature is from 65° C. to 80° C.

7. The method according to claim 1 wherein the weight ratio of water and a nitrile compound in the nitrile compound or the mixture of water and the nitrile compound is 80:20 to 0:100.

8. A method of producing propylene oxide which comprises reacting propylene, oxygen and hydrogen in a liquid phase in the presence of a titanosilicate catalyst regenerated by contacting titanosilicate deteriorated in catalytic ability with a nitrile compound or a mixture of water and a nitrile compound at a temperature from 25° C. to 200° C. and a noble metal catalyst.

9. The method according to claim 8 wherein the noble metal is palladium, platinum, ruthenium, rhodium, iridium, osmium, gold or an alloy or mixture thereof.

10. The method according to claim 8 wherein the noble metal is palladium.
